(19) United States
(12) Patent Application Publication
(10) Pub. No.: US 2014/0350343 A1
(43) Pub. Date: Nov. 27, 2014

(54) DISPOSABLE ENDOSCOPE

(71) Applicant: OPTIMEDE INC., Guro-gu, Seoul (KR)

(72) Inventor: Hun Tae Kim, Seoul (KR)

(73) Assignee: OPTIMEDE INC., Guro-gu, Seoul (KR)

(21) Appl. No.: 14/345,265

(22) PCT Filed: Sep. 21, 2012
(86) PCT No.: PCT/KR12/07577

§ 371 Date: Mar. 17, 2014

(30) Foreign Application Priority Data
Sep. 22, 2011 (KR) 10-2011-0095757

(51) Int. Cl. A61B 1/00; A61B 1/07; G02B 6/26

(52) U.S. Cl. CPC A61B 1/00103; G02B 6/262; A61B 1/00096; A61B 1/07; A61B 1/00165; G02B 2006/12104
USPC 600/121

(57) ABSTRACT

Provided is a disposable endoscope that enables simple treatment by capturing an image inside a body as a moving picture or photo, and in particular, that enables replacement of a probe portion inserted into the body and that preemptively prevents infection in patients due to repeated usage. The disposable endoscope includes: a main body that is connected to a monitor, and in front of which a light emitting portion and a light receiving portion are arranged in parallel; a light emitting fiber that guides light from the light emitting portion toward an object to be examined; a light receiving fiber that guides light reflected from the object to be examined toward the light receiving portion; and a light adjustment unit that is placed within the protective tube that is adjacent to ends of the light emitting fiber and the light receiving fiber and that adjusts a position of light toward the object to be examined or light reflected from the object to be examined, wherein the light receiving fiber guides light that is reflected from the object to be examined through the light adjustment unit toward the light receiving portion. A probe portion inserted into a body of a patient or a user is used only once so that cross-contamination caused by repeated usage can be prevented in advance. A structure of the disposable endoscope is simple so that costs can be reduced due to reduction in a unit cost of production. A portion in which photographing of a moving picture or photo is substantially performed can be precisely controlled so that a precise diagnosis can be carried out. Photographing of a moving picture or photo with high resolution can be performed using a plurality of mirrors and lenses so that the quality of a patient's treatment or therapy can be improved.

### TECHNICAL FIELD

The present invention relates to an endoscope, and more particularly, to a disposable endoscope that enables simple treatment by capturing an image inside a body as a moving picture or photo, and in particular, that enables replacement of a probe portion inserted into the body and that preemptively prevents infection in patients due to repeated usage.

### BACKGROUND ART

In general, medical endoscopes (hereinafter referred to as endoscopes) are medical instruments that are inserted into a hollow organ or cavity of a body and enable examination of the interior of the hollow organ or cavity of the body without performing direct surgery. Endoscopes have made great contributions toward medical development since being devised by Kussmaul in 1863.

In terms of a development procedure of endoscopes, first generation endoscopes that showed a rough image by combining images formed on each optical fiber through a bundle of optical fibers developed into second generation endoscopes that capture an image of a necessary part using a lens of a small camera, display the captured image on a monitor and display a clearer screen. Thus, second generation endoscopes are mainly used at present.

Also, endoscopes with various functions have been developed and used in many fields.

However, the biggest problem in endoscopes according to the related art is a risk of cross-contamination that occurs when an endoscope that has been used once in a patient's treatment is reused.

That is, in terms of the usage of a conventional endoscope, a probe portion of the endoscope that has been once used is sterilized with a disinfectant and is reused. Thus, when the probe portion of the endoscope is not properly sterilized, the probe portion may cause a fatal disease in the patient when it is reused.

In order to solve this problem, technologies disclosed in Korean Patent Registration No. 1007846390000 illustrated in FIG. 1 and Korean Patent Registration No. 100864986000 illustrated in FIG. 2 have been suggested.

Referring to FIG. 1 which illustrates a prior art, an endoscope includes an endoscope body 100, a light source device 150, and a video system 160. The endoscope body 100 includes an insertion portion 110 that is freely curved so as to be inserted into a cavity of the body, a manipulation portion 120 that enables an examiner to perform various functions, a universal code 130 that extends from the manipulation portion 120, and an endoscope body connector (not shown) that is provided at a front end of the universal code 130.

Referring to FIG. 2 which illustrates a different prior art from the prior art of FIG. 1, a state in which an endoscope is covered with an endoscope cover 20, will be described. When the endoscope is covered with the endoscope cover 20, a convex lens portion 12 of the endoscope 10 and an endoscope body portion 11 having a cylindrical shape with a predetermined radius can be blocked from direct contact with foreign substances and germs inside the body when the endoscope is inserted into the body.

The endoscope cover 20 includes an endoscope lens cover 22 and an endoscope body cover 21. The endoscope lens cover 22 is formed of a polyethylene resin so as to prevent the endoscope lens cover 22 from tearing when force is applied to an outer side of the body as the endoscope covered with the endoscope cover 20 is inserted into the body, and concentrated on the lens portion 12 of the endoscope.

Although the above-described prior arts have their own advantages, commonly, a partial probe portion is covered with a protective wrap, or a replaceable cap is provided at the probe portion, rather than replacing a whole probe portion of the endoscope. Thus, there are technical limitations, such as the need of a post-process including sterilization and incomplete prevention of the risk of causing a disease.

### DISCLOSURE

### Technical Problem

The present invention is directed to providing a disposable endoscope that enables direct replacement of a probe portion inserted directly into a patient's body, i.e., that fundamentally solves a problem of reuse so that a risk of germ contamination that may occur in the patient's treatment can be prevented, in particular, a moving picture or photo with high quality and high resolution can be transmitted to a monitor so that the quality of treatment can be improved.

### Technical Solution

One aspect of the present invention provides a disposable endoscope including: a main body that is connected directly to a monitor or is connected to the monitor via a computer or an image processing device, and in front of which a light emitting portion and a light receiving portion are arranged in parallel; a light emitting fiber that is installed to be detachable from the main body and guides light from the light emitting portion toward an object to be examined; a light receiving fiber that guides light reflected from the object to be examined toward the light receiving portion; a protective tube in which the light emitting fiber and the light receiving fiber are accommodated so as to be protected; and a light adjustment unit that is placed within the protective tube that is adjacent to ends of the light emitting fiber and the light receiving fiber and that adjusts a position of light toward the object to be examined or light reflected from the object to be examined, wherein the light receiving fiber may guide light that is reflected from the object to be examined through the light adjustment unit toward the light receiving portion.

The light adjustment unit may include: a fixing mirror that is fixedly installed in the protective tube, guides light guided through the light emitting fiber downward and guides light reflected from the object to be examined toward the light receiving fiber; a driving unit placed within the protective tube of a lower side of the fixing mirror; and an adjustment mirror, for which axial support, rotation, and adjustment of a predetermined inclination angle are provided by the driving unit, that guides light guided through the fixing mirror toward the object to be examined and that guides light reflected from the object to be examined toward the fixing mirror.

A collimator that is a light receiving lens for directivity and condensing characteristics of light may be placed on an optical path between the fixing mirror and the light emitting fiber and between the fixing mirror and the light receiving fiber.

The driving unit may include a micromirror for rotating the adjustment mirror and a solenoid for adjusting an angle of the adjustment mirror.

The driving unit may be a piezoelectric ceramic interlocked by a predetermined signal.

The driving unit may be a piezoelectric film interlocked by a predetermined signal.

The driving unit may be a two-dimensional (2D) optic micro electronic mechanical system (MEMS) that is interlocked by a predetermined signal.

The driving unit may be a one-dimensional (1D) optic MEMS that is interlocked by a predetermined signal.

One selected from the group consisting of a solenoid, a micromotor, a piezoelectric film, a piezoelectric device, and an MEMS may be used as the driving unit.

A plurality of driving units may be selected from the group consisting of a solenoid, a micromotor, a piezoelectric film, a piezoelectric device, and an MEMS may be used in combination as the driving unit.

The light receiving fiber may guide light that is reflected from the object to be examined and guided through the adjustment mirror and the fixing mirror toward the light receiving portion.

### Effects of the Invention

As described above, in a disposable endoscope according to an exemplary embodiment of the present invention, firstly, a probe portion inserted into a body of a patient or a user is used only once so that cross-contamination caused by repeated usage can be prevented in advance.

Secondly, a structure of the disposable endoscope is simple so that costs can be reduced due to reduction in a unit cost of production.

Thirdly, a portion in which photographing of a moving picture or photo is substantially performed can be precisely controlled so that a precise diagnosis can be carried out.

Fourthly, photographing of a moving picture or photo with high resolution can be performed using a plurality of mirrors and lenses so that the quality of a patient's treatment or therapy can be improved.

### DESCRIPTION OF DRAWINGS

FIGS. 1 and 2 illustrate structures and problems of endoscopes according to the related art.

FIG. 3 illustrates a configuration of a whole endoscope system to which a disposable endoscope is applied according to an exemplary embodiment of the present invention.

FIG. 4 illustrates a configuration of main parts of the disposable endoscope illustrated in FIG. 3.

FIG. 5 illustrates a combined state of adjustment mirrors.

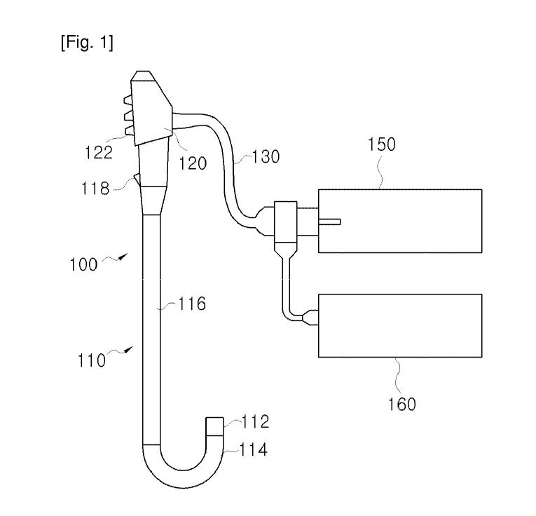

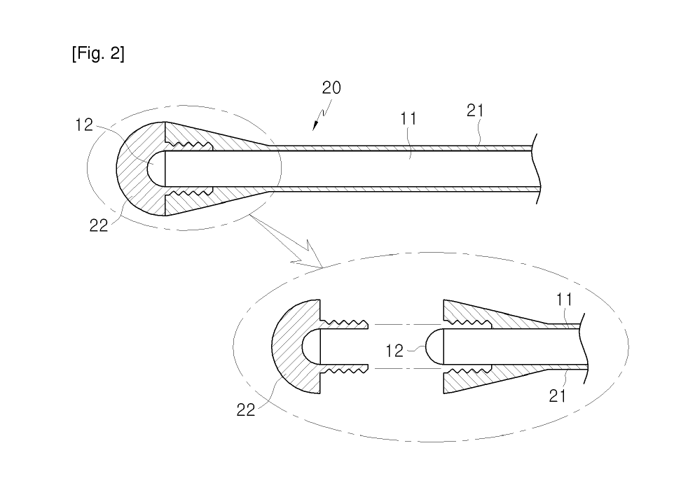

### MODES OF THE INVENTION

Hereinafter, exemplary embodiments of the present invention will be described in detail.

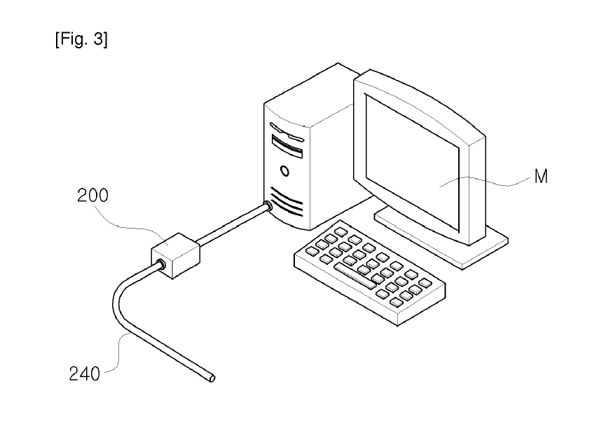

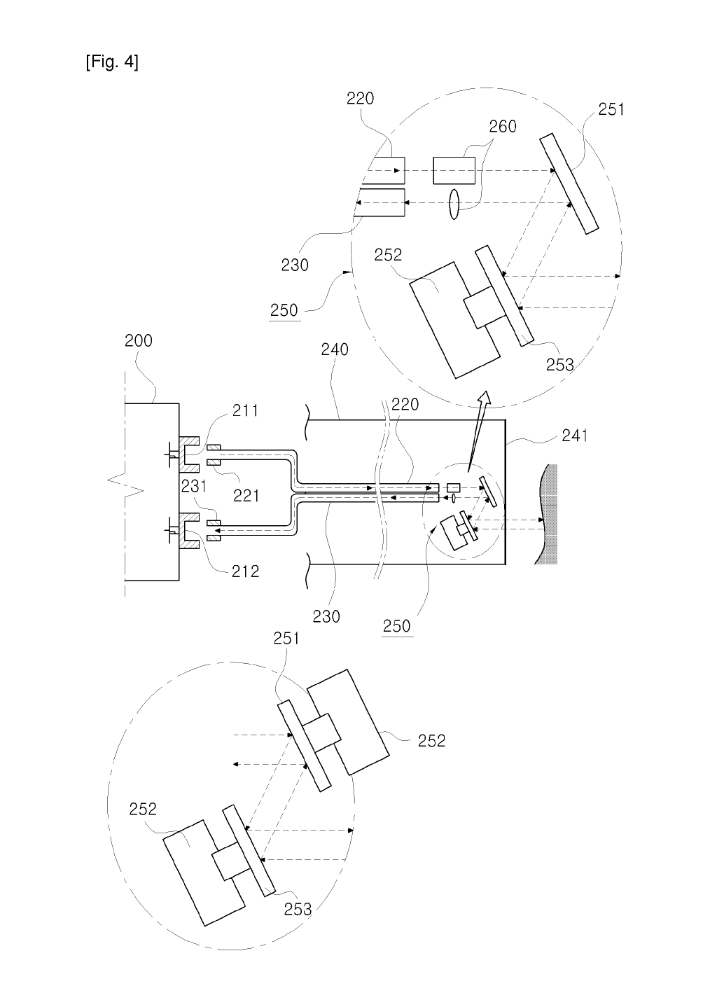

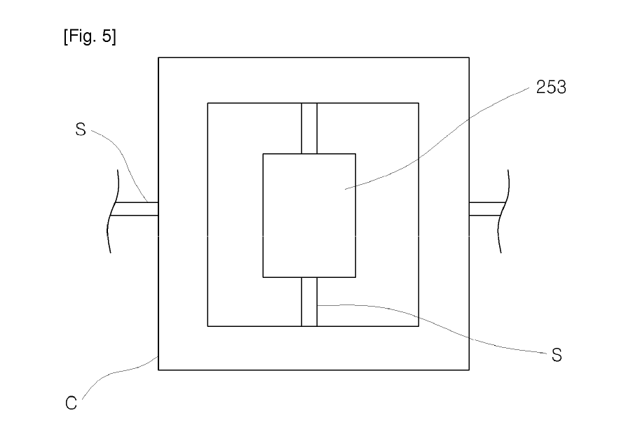

FIG. 3 illustrates a configuration of a whole endoscope system to which a disposable endoscope is applied, according to an exemplary embodiment of the present invention, and FIG. 4 illustrates a configuration of main parts of the disposable endoscope illustrated in FIG. 3, and FIG. 5 illustrates a combined state of adjustment mirrors.

First, as illustrated, a disposable endoscope according to an exemplary embodiment of the present invention is largely configured of a main body 200, a light emitting fiber 220, a light receiving fiber 230, a protective tub 240, and a light adjustment unit 250.

First, the main body 200 is connected directly to a monitor M or is connected to the monitor M via a computer or an image processing device (not shown), and a light emitting portion 211 and a light receiving portion 212 that are portions for transmitting an image are arranged in front of the main body 200 in parallel.

In this case, a laser diode or a light emitting diode (LED) may be used as a light source of the light emitting portion 211.

Also, an image sensor or a photodiode may be used as the light receiving portion 212.

The light emitting fiber 220 is installed to be detachable from the light emitting portion 211 of the main body 200 and performs a function of guiding light on the light emitting portion 211 toward an object to be examined.

In this case, light on the light emitting portion 211 may be focused using a condensing lens or a lens having a similar function to the condensing lens and may be effectively transmitted to the light emitting fiber 220.

The light receiving fiber 230 is also installed to be detachable from the light receiving portion 212 of the main body 200 and performs a function of guiding light reflected from the object to be examined toward the light receiving portion 212. The light receiving fiber 230 may guide reflected light toward the light receiving portion 212 from the object to be examined using the light adjustment unit 250 that will be described later. That is, the light receiving fiber 230 may guide light on the light emitting portion 211 that passes through the light emitting fiber 220, that is transmitted to the object to be examined via a fixing mirror 251 and an adjustment mirror 253 and that is reflected and is re-reflected in the order of the adjustment mirror 253 and the fixing mirror 251, toward the light receiving portion 212. Thus, the light receiving fiber 230 according to the present invention guides only scattered light reflected through the adjustment mirror 253 and the fixing mirror 251, among scattered light reflected from the object to be examined at various angles, toward the light receiving portion 212. Thus, an angle of light guided toward the light receiving portion 212 is substantially limited. Thus, the disposable endoscope according to the present invention receives scattered light at a single angle, and does not receive a substantial portion of disturbance light and/or re-reflected scattered light that may be present via the light receiving portion 212 because the angle is not right. Thus, the disposable endoscope can obtain precise light data without being affected by the disturbance light and/or re-reflected scattered light, thereby obtaining an image with clear quality.

Light reflected from the object to be examined that arrives via the light receiving fiber 230 may be focused using a condensing lens or a lens having a similar function to the condensing lens and may be effectively transmitted to the light receiving portion 212.

In this case, when the light emitting fiber 220 and the light receiving fiber 230 are installed to be detachable from the light emitting portion 211 and the light receiving portion 212 of the main body 200, sleeves 221 and 231 may be provided at ends of the light emitting fiber 220 and the light receiving fiber 230. The sleeves 221 and 231 may be formed of zirconia so as to prevent occurrence of scratching or a risk of damage in a detachment procedure.

The protective tube 240 is formed of rubber or a synthetic resin, i.e., a material that is not harmful to the body. The protective tube 240 has a structure in which the light emitting fiber 220 and the light receiving fiber 230 are accommodated so as to be protected. Also, a distal end 241 of the protective tube 240 may be formed of a material through which light emitted from the light emitting fiber 220 or light incident through the light receiving fiber 230 may pass. The distal end 241 of the protective tube 240 may be formed of any material through which light may pass.

The light adjustment unit 250 is placed at an inner side of the protective tube 240 in which the light emitting fiber 220 and the light receiving fiber 230 are accommodated so as to be protected. The light adjustment unit 250 performs a function of adjusting a position of light moving toward or reflected from the object to be examined.

In a detailed structure of the light adjustment unit 250, the fixing mirror 251 is installed in the protective tube 240 that is adjacent to an end of the light emitting fiber 220 so as to reflect light guided through the light emitting fiber 220 downward and simultaneously to reflect light reflected from the object to be examined, through the light receiving fiber 230.

A driving unit 252 that axially supports, rotates and adjusts the inclination of the adjustment mirror 253 to a predetermined angle, and the adjustment mirror 253 are placed at a lower side of the fixing mirror 251.

That is, the driving unit 252 rotates and adjusts the inclination angle of the adjustment mirror 253, and the adjustment mirror 253 performs a function of reflecting light reflected through the fixing mirror 251 toward the object to be examined and simultaneously reflecting light reflected from the object to be examined, through the fixing mirror 251.

However, embodiments of the present invention are not limited thereto, and the fixing mirror 251 may also be installed to be adjustable by the driving unit 252.

When the adjustment mirror 253 is adjusted in this way, the adjustment mirror 253 is supported on a case C with a shaft S, as illustrated in FIG. 5. Although not shown, when the case C is supported on the protective tube 240 or other fixing units with the shaft S, this interlocking can be more efficiently performed.

In this case, various types of driving units 252 that drive the adjustment mirror 253, such as a micromotor that rotates the adjustment mirror 253 and a solenoid that adjusts an angle of the adjustment mirror 253, may be used.

Also, a piezoelectric ceramic that is interlocked by a predetermined signal, or a piezoelectric film may be used as the driving unit 252.

Meanwhile, a collimator 260 that is a light receiving lens for improving directivity and condensing characteristics of light may be placed on an optical path between the fixing mirror 251 and the light emitting fiber 220 and between the fixing mirror 251 and the light receiving fiber 230.

However, embodiments of the present invention are not limited thereto, and a plurality of lenses may be further provided in addition to the collimator 260, so as to improve directivity and light receiving characteristics of light.

Another embodiment of the light adjustment unit 250 will be described below.

First, the fixing mirror 251 rotates and vibrates a mirror in one of an x-axis direction and a y-axis direction at a predetermined angle, and the adjustment mirror 253 rotates and vibrates the mirror in the other direction so that the mirror can be moved separately in the x-axis and the y-axis directions, and emitted light can be radiated onto the object to be examined or light reflected from the object to be examined can be received. In this way, rotation and vibration about two axes is performed so that light having a predetermined area can be received and an image can be made and checked.

A two-dimensional (2D) optic micro electronic mechanical system (MEMS) or a one-dimensional (1D) MEMS that is interlocked by a predetermined signal may be used as the driving unit 252 for the light adjustment unit 250.

In this case, 2D means driving in both directions of the x-axis and the y-axis, and 1D means driving in one direction.

Consequently, one may be selected from the group consisting of the solenoid, the micromotor, the piezoelectric film, the piezoelectric device, and the MEMS described above and used as the driving unit 252 of the light adjustment unit 250, or a plurality of driving units may be selected therefrom and used in combination as the driving unit 252 of the light adjustment unit 250.

In another embodiment of the present invention, referring to FIG. 4, positions of the fixing mirror 251 and the adjustment mirror 253 may be exchanged. That is, the adjustment mirror 253 may be provided in the position of the fixing mirror 251 of FIG. 4, and the fixing mirror 251 may be provided in the position of the adjustment mirror 253 of FIG. 4. If an angle adjustment range of the adjustment mirror 253 is narrow, the fixing mirror 251 having a convex mirror shape is provided in the position of the adjustment mirror 253 of FIG. 4 even when the range of the angle adjusted by the adjustment mirror 253 is small, and a scanning range of the disposable endoscope according to the present invention can be enlarged.

Operations and effects of the disposable endoscope having the above structure according to the present invention will be described below.

First, when light is radiated from the light emitting portion 211 of the main body 200, i.e., a laser diode or an LED, light is guided through the light emitting fiber 220 that is combined with the light emitting portion 211 through the sleeve 221, passes through the fixing mirror 251 that is close to the end of the light emitting fiber 220 and the adjustment mirror 253 placed at the lower side of the fixing mirror 251 and then is radiated onto the object to be examined.

In this case, any one or each of the fixing mirror 251 and the adjustment mirror 253 is interlocked by the driving unit 252 and guides light to a precise examination position.

Light reflected from the object to be examined passes through the adjustment mirror 253 and the fixing mirror 251 in a reverse order, guided toward the light receiving fiber 230, and simultaneously guided toward the light receiving portion 212 of the main body 200, for example, an image sensor or a photodiode, and information regarding the light can be transmitted to the monitor M via the main body 200 so that a part to be examined can be checked with the naked eye in a moving picture or photo.

In this case, the transmitted moving picture or photo is transmitted via the collimator 260 so that the moving picture or photo with high quality can be checked with the naked eye.

When treatment has been completed in this way, a combination of the sleeves 221 and 231 of a probe portion, i.e., the light emitting fiber 220 and the light receiving fiber 230, is released from the main body 200, and a whole probe portion is discarded, and thus the endoscope cannot be reused for a next patient's treatment.

That is, when new probe portions including a protective tube, a light emitting fiber, a light receiving fiber, and a light adjustment unit are used in combination, a patent's treatment can be performed without risk of cross-contamination.

Consequently, disposable probe portions are provided and are replaced with new ones so that treatment using an endoscope can be safely performed and treatment efficiency and quality can be improved.

While the invention has been shown and described with reference to certain exemplary embodiments thereof, it will be understood by those skilled in the art that various changes in form and details may be made therein without departing from the spirit and scope of the invention as defined by the appended claims.

1. A disposable endoscope comprising:
a main body (200) that is connected directly to a monitor or is connected to the monitor via a computer or an image processing device, and in front of which a light emitting portion (211) and a light receiving portion (212) are arranged in parallel;
a light emitting fiber (220) that is installed to be detachable from the main body (200) and guides light from the light emitting portion (211) toward an object to be examined;
a light receiving fiber (230) that guides light reflected from the object to be examined toward the light receiving portion (212);
a protective tube (240) in which the light emitting fiber (220) and the light receiving fiber (230) are accommodated so as to be protected; and
a light adjustment unit (250) that is placed within the protective tube (240) that is adjacent to ends of the light emitting fiber (220) and the light receiving fiber (230) and that adjusts a position of light toward the object to be examined or light reflected from the object to be examined,
wherein the light receiving fiber (230) guides light that is reflected from the object to be examined through the light adjustment unit (250) toward the light receiving portion.

2. The disposable endoscope of claim 1, wherein the light adjustment unit (250) comprises:
a fixing mirror (251) that is fixedly installed in the protective tube (240), guides light guided through the light emitting fiber (220) downward and guides light reflected from the object to be examined toward the light receiving fiber (230);
a driving unit (252) placed within the protective tube (240) of a lower side of the fixing mirror (251); and
an adjustment mirror (253), for which axial support, rotation, and adjustment of a predetermined inclination angle are provided by the driving unit (252), that guides light guided through the fixing mirror (251) toward the object to be examined and guides light reflected from the object to be examined toward the fixing mirror (251).

3. The disposable endoscope of claim 2, wherein a collimator (260) that is a light receiving lens for directivity and condensing characteristics of light is placed on an optical path between the fixing mirror (251) and the light emitting fiber (220) and between the fixing mirror (251) and the light receiving fiber (230).

4. The disposable endoscope of claim 2, wherein the driving unit (252) comprises a micromirror for rotating the adjustment mirror (253) and a solenoid for adjusting an angle of the adjustment mirror (253).

5. The disposable endoscope of claim 2, wherein the driving unit (252) is a piezoelectric ceramic interlocked by a predetermined signal.

6. The disposable endoscope of claim 2, wherein the driving unit (252) is a piezoelectric film interlocked by a predetermined signal.

7. The disposable endoscope of claim 2, wherein the driving unit (252) is a two-dimensional (2D) optic micro electronic mechanical system (MEMS) that is interlocked by a predetermined signal.

8. The disposable endoscope of claim 2, wherein the driving unit (252) is a one-dimensional (1D) optic MEMS that is interlocked by a predetermined signal.

9. The disposable endoscope of claim 2, wherein one selected from the group consisting of a solenoid, a micromotor, a piezoelectric film, a piezoelectric device, and an MEMS is used as the driving unit (252).

10. The disposable endoscope of claim 2, wherein a plurality of driving units (252) are selected from the group consisting of a solenoid, a micromotor, a piezoelectric film, a piezoelectric device, and an MEMS, and are used in combination as the driving unit (252).

11. The disposable endoscope of claim 2, wherein the light receiving fiber (230) guides light that is reflected from the object to be examined and guided through the adjustment mirror (253) and the fixing mirror (251) toward the light receiving portion (212).
